(19) United States
(12) Patent Application Publication
(10) Pub. No.: US 2014/0206048 A1
(43) Pub. Date: Jul. 24, 2014

(54) COMPOSITIONS AND METHODS FOR BIOFERMENTATION OF OIL-CONTAINING FEEDSTOCKS

(71) Applicant: Glycos Biotechnologies, Inc., Houston, TX (US)

(72) Inventors: Daniel J. Monticello, The Woodlands, TX (US); Ryan Black, Spring, TX (US); Paul Campbell, Houston, TX (US)

(73) Assignee: Glycos Biotechnologies, Inc., Houston, TX (US)

(21) Appl. No.: 14/161,244

(22) Filed: Jan. 22, 2014

Related U.S. Application Data
(63) Continuation of application No. PCT/US2012/048694, filed on Jul. 27, 2012.
(60) Provisional application No. 61/512,748, filed on Jul. 28, 2011.

(51) Int. Cl. C12P 7/42

(52) U.S. Cl. CPC C12P 7/42
USPC 435/135; 435/198; 435/252.1; 435/255.2; 435/348; 435/325; 435/410; 435/252.8; 435/255.1; 435/252.5; 435/136; 435/141; 435/144; 435/157; 435/163; 435/158; 435/146; 435/183; 435/145; 435/167; 435/252.33; 252/182.12

(57) ABSTRACT

The present invention provides compositions and methods for the use of oil-containing materials as feedstocks for the production the bioproducts by biofermentation. In one preferred embodiment, surfactants are not used in compositions and the methods of the invention. In one preferred embodiment the oil-containing feedstocks are the by-products of other industrial processes including microbial, plant and animal oil processing.

### RELATED APPLICATIONS

This application is a Continuation Application of International Application No. PCT/US2012/048694, which designated the United States and was filed on Jul. 27, 2012, published in English, which claims the benefit of U.S. Provisional Application No. 61/512,748, filed on Jul. 28, 2011. The entire teachings of the above applications are incorporated herein by reference.

### BACKGROUND OF THE INVENTION

There is a general interest in producing biochemicals and other bioproducts from renewable, sustainable non-food feedstocks. Plant oils whose major component are Tri-Acyl Glycerides (“TAGs”) have been considered for several years as an alternative carbon source instead of feedstocks that are also used for foods such as corn, for the production of biochemicals, particularly polyhydroxyalkanoates such as polyhydroxybutryate, since the starting and finished materials are both highly reduced. However, the problem is that these substrates are not soluble in water and pose major practical problems and kinetic (mass transfer) limitations for potential industrial fermentations.

A recent publication by researchers at MIT (Budde et al. (29 Jan. 2011) Appl. Microbiol. Biotechnol. DOI 10.1007/s00253-011-3102-0) describes some of the difficulties associated with using plant oils for fermentations. The MIT researchers noted that the addition of insoluble plant oil to aqueous growth medium creates a heterogeneous mixture, in which the low-density oil is concentrated at the top of the vessel, collects on the walls of the vessel and causes the feedstock to be unavailable to the cells.

In addition to these practical difficulties there are significant mass transfer limitations associated with the use of insoluble substrates such as plant oils and hydrocarbons, since the rate of dissolution in the aqueous phase is proportional to the size of the oil droplet. As the material coalesces the droplets get larger and the total surface area decreases. These problems are especially acute with oils that are semi solids (commonly referred to as “fats” in their semi solid state) at operating temperatures, as is the case with most TAGs including palm oil (PO) and palm oil production by-products such as palm acid oil (PAO) and palm fatty acid distillate (PFAD). The prior art has addressed this problem with the use of surfactants to stabilize the oil-in-water emulsion and the droplet size but this is not a practical solution at the industrial scale since these surfactants are expensive.

Others have suggested the use of fatty acids derived from TAGs as fermentation feedstocks and demonstrated the production of biochemical's (e.g., PCT Publication: WO 2009/078973 A2; Dellomonaco et al., (August 2010) Appl. Environ. Microbiol., 76(15): 5067-5078; Rahman al., (2002) Biotechnol Prog. 18:1277-1281) but all the published results require the uses of a surfactant to maintain the oil-in-water emulsion. The prior art typically used 0.5 g/l fatty acid and 0.2 g/l surfactant mixtures, which would be extremely costly in large scale industrial process practice.

It would be desirable to use these abundant oil-containing materials including by-products of other industrial processes as renewable, sustainable, non-food feedstocks in biofermentation processes for the production of bioproducts and biochemicals in a cost effective manner.

### SUMMARY OF THE INVENTION

The present invention provides compositions and methods for the use of oil-containing materials as feedstocks for the production of the bioproducts by biofermentation. In one preferred embodiment, surfactants are not used in the compositions and the methods of the invention. In one preferred embodiment the oil-containing feedstocks are the by-products of other industrial processes including microbial, plant and animal oil processing. In another embodiment, the oils within the oil-containing feedstocks are atomized at a temperature that is above the melting temperature of the oils, prior to their addition to and use in fermentation media.

In another embodiment, the invention relates to a method of forming a dispersion or suspension by heating a substance, preferably a semi-solid substance or fat, above its melting point, followed by atomizing and mixing with a media. In one embodiment, a semi-solid substance or a fat containing substance, for example PFAD, is heated above its melting point and atomized. In one embodiment, the invention relates to heating a composition comprising a semi-solid or fat substance above the melting temperature of one or more of the semi-solid or fat substances and atomizing the heated composition, followed by addition of the atomized composition to a medium to form a dispersion or suspension. The atomized semi-solid or fat substance, or a composition comprising the semi-solid or fat substance is added to a medium to form a dispersion or suspension, preferably said medium is a fermentation media. In a preferred embodiment, PFAD is heated above its melting point, for example a temperature above about 45° C., preferably above 48° C., preferably above 50° C., preferably above 52° C., preferably above 55° C., preferably above 58° C., preferably above 60° C., preferably above 70° C., preferably above 80° C., and atomized followed by addition to an aqueous medium to result in a feedstock for biofermentation. A composition containing PFAD can be heated to a temperature between about 45° C. and about 150° C., preferably between about 50° C. and about 125° C., followed by atomization and addition to an aqueous medium such as fermentation medium.

### BRIEF DESCRIPTION OF THE DRAWINGS

FIG. 1 shows growth of E. Coli FAO1 cells on PFAD.

FIG. 2 is a chromatogram showing the production of acetate, 1,3-PDO and other products from PFAD by B. coagulans NRRL NRS-58.

FIG. 3 is a chromatogram showing the production of succinate and other products by B coagulans NRRL B-1167.

FIGS. 4 A-D are photographs showing dispersion and diameter of atomized PFAD in cold water.

FIG. 5 is a line graph showing cell growth on PFAD and mevalonate production over 48 hours.

FIG. 6 is a line graph showing PFAD dissimilation during cell culture over 48 hours.

FIG. 7 is a line graph showing PFAD dissimilation and cell growth over two weeks.

FIG. 8 is a line graph showing cell growth on PFAD over 72 hours.

FIG. 9 is a line graph showing mevalonate production from PFAD over 72 hours.

FIG. 10 is a line graph showing residual PFAD fatty acid levels from cell culture over 72 hours.

### DETAILED DESCRIPTION OF THE INVENTION

In one embodiment, the invention provides a method of producing bioproducts from oil-containing feedstocks comprising fats, oils and fractions thereof, preferably without the use of surfactants. The method comprises providing a feedstock comprising fats, oils and fractions thereof; heating the feedstock to a temperature that is above the melting point of the fats, oils and fractions thereof contained in the feedstock; adding the heated feedstock to the fermentation media while mixing the fermentation media wherein the fermentation media comprises at least one biocatalyst capable of fermentation in the presence of the feedstock, wherein the feedstock forms a stable dispersion upon addition to the fermentation media; fermenting the feedstock in the fermentation media while mixing the fermentation media to produce at least one bioproduct; and optionally collecting crude bioproduct from the fermentation medium. In one preferred embodiment, the fermentation media comprising the heated feedstock is also preferably maintained at a temperature that is above the melting point of the oils contained in the feedstock throughout the fermentation process. In a preferred embodiment, the fermentation media and feedstock are free of surfactants. In another embodiment, the oils within the oil-containing feedstocks are atomized at a temperature that is above the melting temperature of the oils, prior to their addition to and use in fermentation media. In a preferred embodiment, the oil containing feedstock is heated to greater than about 45° C., preferably above 48° C., preferably above 50° C., preferably above 52° C., preferably above 55° C., preferably above 58° C., preferably above 60° C., preferably above 70° C., preferably above 80° C., above about 100° C., atomized, added with optional stirring to fermentation media that is at a temperature below the melting point of the fats, such as between about 15° C. to about 45° C., and fermented at a normal fermentation temperature, such as between about 15° C. to about 45° C.

In another embodiment, the invention relates to a method of forming a dispersion or suspension by heating a substance, preferably a semi-solid substance or fat above its melting point, followed by atomizing and mixing with a media. In one embodiment, a semi-solid substance or a fat containing substance, for example PFAD or PAO, is heated above its melting point and atomized. In one embodiment, a composition containing a semi-solid or fat substance such as PFAD or PAO, which is feedstock for biofermentation, is heated above the melting point of PFAD or PAO and atomized. In one embodiment, substantially all solid substances within the composition are melted to a level allowing for their atomization. The atomized semi-solid or fat substance is added to a medium, preferably an aqueous medium, to form a dispersion or suspension. In a preferred embodiment, the aqueous medium is suitable for biofermentation. In a preferred embodiment, PFAD is heated above its melting point, for example above about 45° C., preferably above 48° C., preferably above 50° C., preferably above 52° C., preferably above 55° C., preferably above 58° C., preferably above 60° C., preferably above 70° C., preferably above 80° C., preferably above 100° C., and atomized followed by addition to an aqueous medium such as a fermentation medium. The aqueous medium can be stirred upon addition of the atomized semi-solid or fat substance to improve the uniformity of the suspension or dispersion. In a more preferred embodiment, the atomized substance, for example PFAC, is added with heavy stirring to fermentation media that is at a temperature below the melting point of the fats, such as between about 15° C. to about 45° C., and fermented at a normal fermentation temperature, such as between about 15° C. to about 45° C.

In one embodiment, the invention provides a method of producing bioproducts from oil-containing feedstock comprising fats, oils and fractions thereof, preferably without the use of surfactants. The method comprises providing a feedstock comprising fats, oils and fractions thereof wherein at least a portion of the fats, oils and fractions thereof are in a form which is at least semi solid; adding the feedstock to fermentation media and mixing the fermentation media, wherein the fermentation media comprises at least one biocatalyst capable of fermentation in the presence of the feedstock, wherein the fermentation media is at a temperature above the melting point of the fats, oils and fractions thereof contained in the feedstock, and wherein the feedstock forms a stable dispersion upon addition to the fermentation media fermenting the feedstock in the fermentation media to produce at least one bioproduct; and optionally collecting crude bioproduct from the fermentation medium.

In a further embodiment, the invention provides a method of producing bioproducts from oil-containing feedstock comprising fats, oils and fractions thereof, preferably without the use of surfactants. The method comprises providing a feedstock comprising fats, oils and fractions thereof wherein at least a portion of the fats, oils and fractions thereof are in a form which is at least semi solid; adding the feedstock to fermentation media and mixing the fermentation media, wherein the fermentation media comprises at least one biocatalyst capable of fermentation in the presence of the feedstock, wherein the fermentation media is at a temperature below the melting point of the fats, oils and fractions thereof contained in the feedstock, and wherein the feedstock forms a stable dispersion upon addition to the fermentation media fermenting the feedstock in the fermentation media to produce at least one bioproduct. In a preferred embodiment, the oil-containing feedstock is heated above about 45° C., preferably above 48° C., preferably above 50° C., preferably above 52° C., preferably above 55° C., preferably above 58° C., preferably above 60° C., preferably above 70° C., preferably above 80° C., preferably above 100° C. and atomized, followed by addition with optional stirring to an aqueous medium such as a fermentation media that is at a temperature below the melting point of the fats, such as between about 15° C. to about 45° C., and fermented at a normal fermentation temperature, such as between about 15° C. to about 45° C.

A variety of atomization devices and nebulization devices can be used to atomize the heated fat or semi-solid substance or composition containing fat or semi-solid substance. The atomization devices can heat and atomize or heated substances in liquid form can be provided that is atomized. Preferred concentrations of PFAD for atomization are at least about 0.5 g/ml, and preferably at least about 0.7 g/ml, and preferably at least about 0.9 g/ml. A preferred method of atomization is spray congealing.

Spray congealing includes the atomization of a fluid into an environment (either vapor or liquid) maintained at a temperature below the fluid's melting point. The atomization leads to the formation of molten droplets which then solidify upon cooling, producing the final microparticles. In pharmaceutical applications of spray congealing atomization can be obtained via a range of devices; the pneumatic nozzles (also called two fluid or air nozzle), the rotary or centrifugal atomizers, and the ultrasonic atomizers. Commonly employed atomizers are the pneumatic nozzles, which can employ either by internal or external mixing. The choice of nozzle may influence the properties and performance of micro-particles prepared by spray congealing. (Passerini et al., Solid Lipid Microparticles Produced by Spray Congealing: Influence of the Atomizer on Microparticle Characteristics and Mathematical Modeling of the Drug Release. JOURNAL OF PHARMACEUTICAL SCIENCES, VOL. 99, NO. 2, FEBUARY 2010, 916-931).

Preferred PFAD concentrations within fermentation media are at least about 2 g/L, and preferably 3 g/L, and preferably 4 g/l, and preferably greater than 4 g/L, such as between 4.1 g/L and 10 g/L.

As used herein the terms “oil-containing feedstock”, “oil containing materials” or “oil-containing by-products” refers to any materials that contain any combination of fats, oils or fractions thereof regardless of the physical state of the fats and oils in the materials; e.g. the material may be a semi-solid or mostly solid and therefore a “fat”, however the term “oil” will still cover any material in the semi-solid or “fat” state. Fats oils and fractions thereof include but are not limited to, monoglycerides, diglycerides, triglycerides, phosphatides, cerebrosides, sterols, terpenes, fatty alcohols, fatty acids, plant waxes and paraffin waxes. In one embodiment oil-containing feedstocks comprise at least about 20% by weight of an oil, fat or fraction thereof; preferably at least about 30% by weight of an oil, fat or fraction thereof; preferably at least about 50% by weight of an oil, fat or fraction thereof; and preferably at least about 70% by weight of an oil, fat or fraction thereof.

In one embodiment, the invention provides a composition for use in biofermentation comprising a feedstock, wherein the feedstock comprises fats, oils or fractions thereof, and wherein the feedstock is stably dispersed in fermentation media, wherein the fermentation media comprises at least one biocatalyst capable of anaerobic fermentation in the presence of the feedstock and wherein the composition is free of surfactants.

In one preferred aspect of this embodiment, the composition for biofermentation further comprises a lipase. The lipase may be added as a separate reagent to the compositions or if the biocatalyst is an organism, such organism may be suitably genetically engineered to over-express a lipase as is known in the art of molecular biology. Lipases may be used herein for their ability to modify the structure and composition of triglyceride oils, fats, fractions thereof and other oleochemicals in the feedstock, the fermentation media, or both to make them more available to the biocatalyst as substrates. Lipases catalyze different types of triglyceride conversions, such as hydrolysis, esterification and transesterification. Suitable lipases include acidic, neutral and basic lipases, as are well-known in the art such as Candida antarcitca lipase and Candida cylindracea lipase. More preferred lipases are purified lipases such as Candida antarcitca lipase (lipase A), Candida antarcitca lipase (lipase B), Candida cylindracea lipase, and Penicillium camembertii lipase. Lipases may be added in amounts from about 1 to 400 LU/g DS (dry solids), preferably 1 to 10 LU/g DS, and more preferably 1 to 5 LU/g DS.

As used herein “feedstock” generally refers to the material that serves as a substrate for the bioconversion of the material to a desired product by the biocatalyst. In accordance with the present invention, the feedstock comprises oil-containing materials including fats, oils and fractions thereof. The feedstock is preferably combined with the biocatalyst preferably in a biofermenter under conditions suitable for bioconversion of the feedstock to the desired product by the biocatalyst. Preferred feedstocks include those that are by-products from other industrial processes including but not limited to: bio-fuel manufacture, fat saponification, alcoholic beverages manufacture, production of vegetable oils and other processes used in the oleochemicals industry. Industrial processes are those relating to the oil-refining industries which generate by-products such as paraffin waxes.

“Conditions suitable for bioconversion of a feedstock” refers to the material and methods for maintenance and growth of microbial cultures that support the biochemical pathways that are necessary for the biocatalyst to convert a specific feedstock to a specific product. Such materials and methods are well known in the art of microbiology and biofermentation. Consideration must be given to appropriate media, pH, temperature and requirements for fermentation conditions depending on the specific requirements of the microorganism to support bioconversion of the given feedstock. “Media” generally refers to the liquid containing nutrients for culturing the biocatalyst microorganisms.

“Fermentation media” also referred to herein as “fermentation broth”, “beer”, or “fermented liquid”, is the liquid in which the fermentation and bioconversion of the feedstock to product takes place which generally takes place in a biofermenter and includes the feedstock, biocatalyst and associated media. The fermentation liquid may be removed from the biofermenter for selective removal of the desired product or separation of spent biocatalyst (e.g., cell mass of micro-organism), viable biocatalyst, and feedstock that has not undergone bioconversion for reuse as described herein. In addition to an appropriate feedstock, fermentation media contains suitable minerals, salts, cofactors, buffers, and other components, known to those skilled in the art. These supplements must be suitable for the growth of the biocatalyst and promote the biochemical pathway necessary to produce the biofermentation product.

The “biocatalyst” may be any microorganism or relevant portion thereof capable of converting a selected feedstock to a desired product. The biocatalyst can be a whole microorganism, one or more isolated enzymes or any combination thereof. For the purposes of this application, “microorganism” includes one or more eukaryotes or prokaryotes and includes bacteria, yeast or cells from an insect, animal or plant or tissues therefrom. The biocatalyst may be whole microorganisms or in the form of isolated enzyme catalysts. Microorganisms may be genetically engineered to provide optimum bioconversion to the desired product. In one preferred embodiment, the biocatalyst organisms of the present invention are genetically engineered to overexpress lipase as is discussed herein.

In accordance with the invention, it is preferable that the fermentation media to be maintained at a temperature that is above the temperature of the melting point of the fats, oils and fractions thereof contained in the feedstock. Such temperatures aid in maintaining the oil-containing feedstock in a stable dispersion in the fermentation media throughout the fermentation process. Maintaining the stability of the dispersion also aids in separating bioproduct, unused biocatalyst and unused feedstock at the end of the fermentation as will be described herein. In one embodiment fermentation may occur at a temperature below the melting point of the oils comprising the feedstock followed by raising the temperature of the fermentation media to above the melting point of the feedstock at the end of fermentation to aid in the separation step.

In accordance with the invention, other embodiments provide for the atomization of the feedstock at a temperature greater than 100° C. prior to its addition to the fermentation media, wherein the temperature of said fermentation media prior to addition of the heated feedstock is between about 15° C. to about 45° C. In another embodiment, fermentation occurs at a temperature that is below the temperature of the melting point of the fats, oils and fractions thereof contained in the feedstock, such as between about 15° C. to about 45° C., such as about 37° C. Atomization of the feedstock provides for stable and homogeneous dispersion of the oil-containing feedstock within the fermentation media and throughout the fermentation process.

In one embodiment, the invention relates to a method wherein about 0.5 to about 100 part by volume of the heated feedstock is added to about 400 parts by volume of fermentation media in about six to eight hour time intervals of fermentation, and optionally an additional 0.5 to about 100 parts of the heated feedstock is added to the fermentation media at about 32, 48, 52, 56, and 72 hours of fermentation. In a preferred embodiment, the feedstock contains PFAD or PAO.

The optimum temperature for fermentation varies depending on the particular fats and oils contained in the feedstock as well as the biocatalyst used, and whether or not the feedstock is atomized prior to its addition to the fermentation media, but a range of about 25° C. to about 70° C. is generally preferred. Higher fermentation temperatures are preferred in the absence of feedstock atomization, such as temperatures above about 30° C., and preferably above about 40° C. and preferably above about 50° C. In one preferred embodiment, the temperature of the fermentation media throughout the fermentation is maintained at about 37° C. or higher when the feedstock has not been atomized. In one preferred embodiment, the temperature of the fermentation media throughout the fermentation is maintained between about 50° C. and about 70° C. when the feedstock has not been atomized. In a more preferred embodiment, the temperature of the fermentation media, prior to addition of the heated and atomized feedstock and throughout the fermentation process, is maintained at normal fermentation temperatures, such as between about 15° C. to about 45° C., and preferably above about 30° C., and preferably about 37° C.

Preferable biocatalyst microorganisms for fermentation are heat resistant. Preferred microorganisms that are suitable as biocatalysts for use in the present invention include those that are mesophilic and thermophilic. Preferred organisms are able to withstand temperatures in excess of 37° C. or higher and produce the desired product in the presence of the oil-containing feedstocks (e.g., the feedstock is not toxic to the biocatalyst and is a suitable carbon source for biocatalyst). For example, organisms suitable for use in the conversion of glycerol to ethanol include, but are not limited to: wild-type and bioengineered microorganisms described in United States Patent Publication 2009/0186392 such as wild-type E. Coli K12 strains MG1655 (ATCC 700926), W3110 (ATCC 27325), MC4100 (ATCC 35695) and E. coli B (ATCC 11303), enteric bacteria Enterobacter cloacae subsp., cloacae NCDC 279-56 (ATCC 13047) and yeast Saccharaomyces cerevisiae. Other suitable organisms include strains such as wild type Paenibacillus macerans (P. macerans) Northrup strain N234A (=LMG13285=N234A) available from the Belgian Coordinated Collections of Microorganisms (BCCM/LMG, Gent, Belgium); and wild-type P. macerans strains B-394 (NRRL collection Peoria, Ill.) and ATCC 7068 (American Type Culture Collection, Manassas, Va.), Bacillus coagulans, Geobacillus. stearothermophilus, and Geobacillus. thermoglucosidasius. Suitable B. coagulans, G. stearothermophilus and G. thermoglucosidasius strains are available from the Bacillus Genetic Stock Center at The Ohio State University.

The terms “product”, “bioproduct” or “biochemical” refer to any biocatalytically produced product that is produced by the biocatalyst as a result of fermentation of the feedstock. Bioproducts produced in accordance with the present invention include, but are not limited to: organic acids (formic, acetic, propionic, butyric, citric, etc.), alcohols (methanol, ethanol, propanol, propandiols, etc.), hydroxy and dihydroxy acids, vitamins, enzymes, antibiotics, polyhydroxyalkanoate or polyhydroxybutyrate. Bioproducts include but are not limited to ethanol, acetate, succinate, mevalonate, isoprene and butyrate.

The present invention is adaptable to a variety of biofermentation methodologies, especially those suitable for large-scale industrial processes. The invention may be practiced using batch, fed-batch, or continuous processes and can be practiced with a wide variety of fermenter vessels.

The present invention also provides a separation process for recovering various components from a fermentation media in which oil-containing feedstock is stably dispersed. It is difficult to accurately monitor the concentration of hydrocarbons, TAGs or fatty acids (FAs) in a two-phase fermentation because of the discontinuous nature of the suspension. Consequently it is difficult to determine when all the substrate in the reactor is consumed. This can result in feedstock remaining at the end of the fermentation, which must be discarded (expensive) or separated from the fermenter beer (technically difficult).

Separation and reuse of the non-polar material at any time throughout the fermentation process or after completion of the fermentation process is considerably easier if it is in the liquid form, in accordance with the present invention such as by using thermophiles as the biocatalyst in fermentations hotter than about 50° C. during fermentation or alternatively heating the fermentation media to above the melting point of the feedstock after fermentation is completed but prior to the separation step. The hydrocarbons, triacylglycerides or fatty acids can be separated in a coalescing filter system, which allows the two phases to separate and then skims off the surface of the liquid. Alternatively, when the hydrocarbons, triacylglycerides or fatty acids are in the liquid form, the two phases may be separated using hydrocyclones. One advantage of this approach is that the biocatalyst may adhere to the non-polar phase, thus facilitating the separation and potential re-use of the cells that are the biocatalyst.

In one preferred embodiment, the present invention provides compositions and methods for the use of oil-containing materials as feedstock, including those oil containing feedstocks that are the by-products of other industrial processes including microbial, plant and animal oil processing, for the production of bio-based chemicals and other biofermentation products without the use of surfactants. In one preferred embodiment, the feedstock comprises by-products of the industrial processes that produce vegetable oils such as palm oil, coconut oil, soybean oil and palm kernel oil. These processes tend to produce as a by-product, various fatty acid distillates via several routes including but not limited to steam distillation or extraction with various organic solvents.

In the Examples, we demonstrated the use of the by-products of vegetable oil production as a feedstock with a particularly difficult substrate, a by-product of palm oil processing known as palm fatty acid distillate (PFAD).

Palm Oil (PO) is obtained from the fruit of the oil palm tree, which grows well in hot, humid, tropical countries, the main ones being Malaysia and Indonesia. Palm oil is in fact a fat in temperate countries, with a melting point of 33-39° C., iodine value 50-55 and solid fat content about 26%. Its fatty acid composition is based on palmitic acid (44%), oleic acid (39%) and linoleic acid (11%). A major advantage is that unlike hydrogenated oils with the same melting point, it contains no trans fatty acids which are now accepted to be risk factors for heart disease. Crude PO is normally traded on the basis of 5% FFA, but most of the exported PO is RBD (refined, bleached and deodorized) grade with free fatty acid (FFA) of 0.1% max. During transportation to distant countries some deterioration in quality is inevitable, but still this grade is acceptable for consumption without any further treatment, in many countries. In Europe and the USA however it is always given a mild refining treatment.

The main uses of PO are in frying and in the production of margarines, shortenings and vanaspati ghee. The soap industry is also a big user, although with it, the color of the soap is not quite as good as with tallow. On the other hand, its vegetable origin gives it other advantages.

Palm Acid Oil (PAO) is a by-product from the chemical refining of palm oil. It consists mainly of FFA (over 50%) and neutral oil, with 2-3% moisture and other impurities. It is very similar to palm fatty acid distillate (PFAD), but its FFA is generally lower. The main uses of PAO are in animal feeds, in soap making and for distilled fatty acid production. This product is not now produced on any great scale outside Europe, because in Malaysia and Indonesia palm oil is refined by the physical process which gives PFAD rather than PAO.

Palm Fatty Acid Distillate (PFAD) is a by-product from the physical refining of palm oil, which is now the most widely used process in the major producing countries. Its scale of production is large enough to support significant international trade in it. PFAD has very similar composition to palm acid oil (PAO), but it generally has higher FFA (over 70%), the balance being neutral oil and up to 1% moisture and impurities. Its main uses are in animal feeds, including some specialty products, in soap making and in the production of distilled fatty acids. PFAD is produced in much greater volume than PAO.

We have demonstrated the difficulties of using PFADs at normal fermentation temperatures (about 15° C. to about 45° C.). As noted in Budde et al. supra, feedstock comprising vegetable oil-containing material adheres to the sides of the vessel and the agitators is not dispersed in the fluid. We repeated these experiments with PFAD as described in the examples and confirmed that PFAD could not be readily dispersed in the media. However, we have shown that if the material is used at temperatures greater than 50° C., the PFAD melts and is readily and stably dispersed in the medium without the need for surfactants. We have also shown that if the material is melted prior to introduction to broth that is below the melting temperature of the PFAD, the material quickly disperses as very small droplets that do not coalesce as long as agitation is continued. We have further shown that if the PFAD is heated to a temperature of greater than 100° C. and subsequently atomized at said temperature prior to introduction into fermentation broth that is at normal fermentation temperatures (about 15° C. to about 45° C., and thus below the melting temperature of the PFAD), the PFAD material quickly disperses as very small droplets that do not coalesce as long as agitation is continued. We have also demonstrated that PFAD is surprisingly not toxic to standard biocatalyst organisms used in industrial fermentation processes. What follows are non-limiting examples of the present invention.

### EXAMPLES

### Example 1

This Example illustrates the difficulties of using PFAD at normal fermentation temperatures.

2 g of PFAD is added to a beaker of 100 ml water at room temperature. The PFAD remains undispersed and agglomerated even in the presence of stirring.

2 g of PFAD is added to a beaker of 100 ml water at room temperature. The PFAD remains undispersed and agglomerated. The solution is heated above the melting temperature of the PFAD (above about 49° C.), and the PFAD forms a film on top of the water. When actively stirred, the PFAD is well dispersed in the water. When this mixture is cooled to the freezing point of PFAD (about 49° C.) the PFAD remains well dispersed as long as the solution is stirred. When stirring is discontinued, the PFAD is no longer stably dispersed and clumps on the sides of the beaker.

2 g of PFAD is melted prior to being added to a beaker of 100 ml of water at room temperature which is being actively stirred. The PFAD is stably dispersed in the water.

### Example 2

This Example demonstrates the growth of bacteria on PFAD.

E. coli FA01, (strain MG 1655: ATCC 700926) was cultured in 10 ml of sterile minimal media in 50 ml baffled Erlenmeyer flasks. Ingredients for the minimal media were added in the following order without stirring to prevent precipitation as follows: 8.4 ml L-alanine, 6.4 ml L-arginine, 5 ml L-asparagine, 13 ml L-aspartate, 5 ml L-cysteine, 40 ml L-glutamate, 5 ml glutamine, 5 ml glycine, 4.2 ml L-histidine, 10 ml L-isoleucine, 16.4 ml L-leucine, 14 ml L-lysine, 5.2 ml L-methionine, 8.6 ml mg/l L-phenylalanine, 10 ml L-proline, 14 ml L-serine, 8.4 ml L-threonine, 3 ml L-tryptophan*HCl, 5.6 ml L-tyrosine, 12.6 ml L-valine, 10 ml biotin (10 mg/100 ml), 10 ml Thiamine*HCl (10 mg/100 ml), 10 ml nicotinic acid (10 mg/100 ml), 0.1 ml anhydrous CaCl₂ (5%), 0.1 ml FeCl₃*6H₂O (0.05%), 0.1 ml ZnSO₄*7H₂O (5%), 0.1 ml MnCl₂ (10 mM), 100 ml mineral salts solution (10 g of NH₄Cl, 10 g of NaCl, 4 g of MgSO₄ per liter), 50 ml potassium phosphate buffer (125 g of K₂HPO₄ and 30 g of KH₂PO₄ per 500 ml). All stock amino acid solutions for the E. coli defined medium were 1% (wt/vol). The final solution was adjusted to pH 7.3 and filtered sterilized.

Flasks were supplemented with sterile 5 g/l CaCO₃ to maintain a neutral pH in cultures. 20 g/l glucose was added to one flask and served as a positive control. A second flask contained only the minimal medium, which served as a negative control. The third flask contained 0.5% (w/v) PFAD which was prepared as follows: 2.5% (w/v) PFAD and 1% (w/v) Brij-58 was separately autoclaved at 121° C. for 20 minutes. The PFAD/Brij-58 blend was then cooled to room temperature with heavy mixing before being added to the media.

Isolated colonies of E. coli were obtained by streaking cells onto LB agar plates from glycerol stocks. One isolated colony was collected by loop and used to seed each flask. Cultures were incubated at 37° C. with shaking at 135 rpm. Samples were collected for analysis at 0, 24, and 48-hour time-points. Cells were counted under a microscope using a C-Chip hemocytometer (Digital Bio, East Sussex, GB).

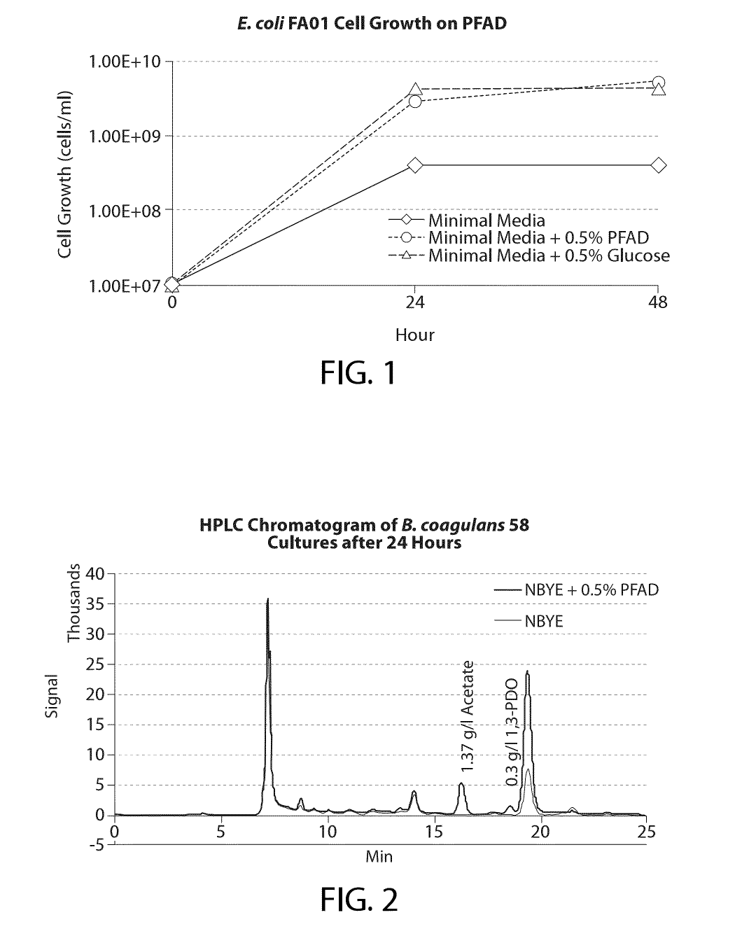

FIG. 1 shows that E. coli grows as well on PFAD as it does on glucose. This demonstrates for the first time that traditional industrially relevant bacteria can grow on PFADs. This was unexpected and surprising because we have evaluated many similar materials that are toxic to the bacteria or do not support growth.

A similar experiment was repeated using the thermophilic bacteria Bacillus coagulans and Geobacillus stearothermophilus and similar enhanced growth was seen. In these experiments the cultures were grown at 55° C., well above the melting temperature of PFAD.

### Example 3

This Example demonstrates that representative thermophiles can grow on PFAD and that PFAD can be used to produce commercially relevant products.

In growth experiments, B. coagulans strains were grown in 10 ml NBYE in 50 ml baffled Erlenmyer flasks. Flasks were supplemented with sterile 5 g/l CaCO₃ to maintain a neutral pH in cultures. 0.05 g of heated, liquid PFAD was added to 10 ml of NBYE for a final concentration of 5 g/l. Each B. coagulans strain was seeded into two cultures: NBYE (control) and NBYE+5 g/l PFAD.

Isolated colonies of B. coagulans were obtained by streaking cells onto NBYE agar plates from glycerol stocks. One isolated colony was collected by loop and used to seed each flask. Cells were allowed to grow for one hour before PFAD was added.

Cultures were grown at 55° C. in a water bath with shaking at 150 rpms for 24 hours.

In product production experiments, B. coagulans cultures were grown in NBYE (5 g/l Difco Nutrient Broth, 20 g/l Yeast Extract, 1.5 g/l NaCl).

Cultures were analyzed for liquid fermentation products by injecting 10 μL samples on to a HPLC (LC-10AD vp, Shimadzu, Kyoto, Japan) using a Rezex ROA-Organic Acid H⁺ (8%) column (Phenomenex, Torrance, Calif.) at 65° C., with a 2.5 mM H₂SO₄ mobile phase (isocratic, 0.6 mL/min), and ultraviolet (UV, SPD-10A vp, 280 nm) and refractive index detectors (RID-10A). All samples were filtered through a 0.22 μm polyvinylidene fluoride syringe filter (Millipore) prior to HPLC analysis.

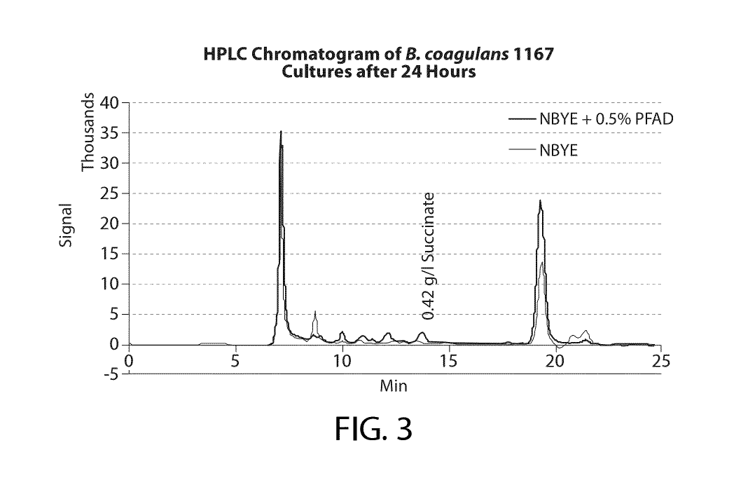

Using similar experimental conditions, other important biochemical were also made by the thermophiles grown at 55° C. This is illustrated in FIGS. 2 and 3. B coagulans NRRL NRS B-58 produced several discrete products, including acetate and 1,3-propapediol. FIG. 2 shows the production of acetate, 1,3-PDO and other products from PFAD by B. coagulans NRRL NRS-58.

FIG. 3 shows the production of succinate and other products from PFAD by B. coagulans NRRL B-1167.

Similar growth experiments were conducted with Geobacillus stearothermophilus strains NRRL B-1102 and NRRL B-4419. These thermophiles also grew well on the dispersed PFAD. This demonstrates for the first time that traditional industrially relevant thermophilic industrial bacteria can grow on PFADs and produce industrially relevant bioproducts.

### Example 4

This example illustrates how atomized PFAD can be used at normal fermentation temperatures, and illustrates one way we can use our PFAD melting strategy.

PFAD is first heated to greater than 100° C. 1.6 ml, or 1.6 g, of heated PFAD is injected through a hot, 26-gauge needle (preheated to greater than 100° C.) into 400 ml of media under normal fermentation temperatures (room temperature to about 45° C.) and while under heavy stirring, for a concentration of 4 g/L PFAD.

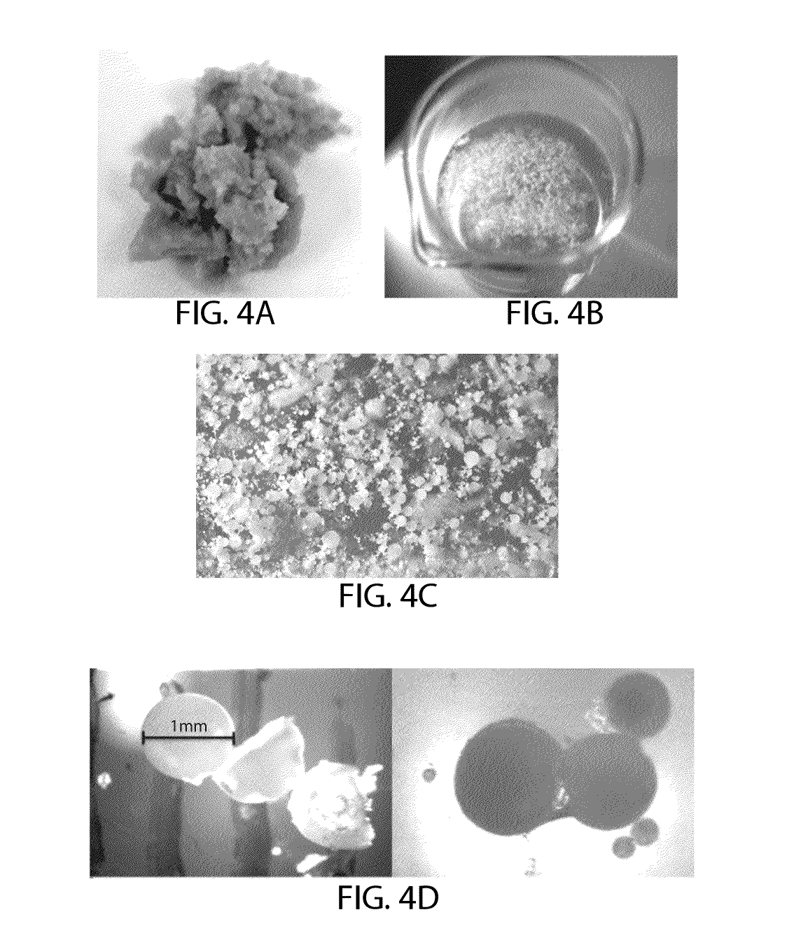

FIG. 4A shows solid non-atomized PFAD. By injecting hot PFAD through a 26-gauge needle into cold water, we are able to homogeneously disperse the PFAD in solution in small spheres of PFAD about 1 mm in diameter (FIG. 4B-D). Active stirring helps to prevent the PFAD spheres from agglomerating. Atomization of PFAD into small 1 mm spheres increases the surface area, and thus the solubility, of 1.6 g of PFAD.

### Example 5

This example demonstrates the growth of bacteria and the production of mevalonate, a precursor for isoprene, from PFAD.

E. coli FA01, (strain MG 1655: ATCC700926), transformed with pGB 1008 (rep_{p15A}, Cm^{R}, tetR, P_{LtetO-1}, mvaES_{Ef,optEc}, T1), was cultured in 400 ml of sterile minimal media in ½L bioreactors. Ingredients for the minimal media were as follows: 660 mg/l ammonium sulfate, 1.2 g/l sodium phosphate dibasic, 3 g/l ammonium chloride, 0.25 g/l potassium sulfate, 4 g/l magnesium chloride hexahydrate, 30 mg/l iron sulfate heptahydrate, 700 mg/l calcium chloride dihydrate, 0.173 mg/l sodium selenite, 0.004 mg/l ammonium molybdate tetrahydrate, 0.025 mg/L boric acid, 0.007 mg/l cobalt chloride hexahydrate, 0.003 mg/l copper (II) sulfate pentahydrate, 0.016 mg/l manganese chloride tetrahydrate, 0.003 mg/l zinc sulfate heptahydrate.

The minimal media was supplemented with 4 g/l hot PFAD that was atomized through a 26-gauge needle. We cultured FA01 at 37° C. and at pH 6.3 maintained with 5M NaOH. This experiment was under microaerobic conditions with an oxygen transfer rate coefficient of k_{L}a=60^{hr-1}. 20 ug/ml chloramphenicol was added to maintain selection of FA01 cells transformed with the pGB1008 plasmid. Plasmid pGB1008 was induced with 100 ug/L anhydro tetracycline.

Isolated colonies of FA01 were obtained by streaking cells onto LB agar plates from glycerol stocks. One isolated colony was collected by loop and used to seed 40 ml LB in a 250 ml baffle shake flask, which served as precultures for experiments. 20 ug/ml chloramphenicol was added to the shake flasks to maintain selection of FA01 cells transformed with the pGB 1008 plasmid. Precultures grew overnight at 37° C. with shaking at 175 rpm. Grown precultures were washed in sterile minimal medium salts before being added to the ½L bioreactors with sufficient cells for a start OD₆₀₀ of 0.1.

For general metabolites, cultures were analyzed for liquid fermentation products by injecting 10 μL samples on to a HPLC (LC-10AD vp, Shimadzu, Kyoto, Japan) using a Rezex ROA-Organic Acid H⁺ (8%) column (Phenomenex, Torrance, Calif.) at 65° C., with a 2.5 mM H₂SO₄ mobile phase (isocratic, 0.6 mL/min), and ultraviolet (UV, SPD-10A vp, 280 nm) and refractive index detectors (RID-10A). All samples were filtered through a 0.22 μm polyvinylidene fluoride syringe filter (Millipore) prior to HPLC analysis.

Fatty acids were analyzed using a HPLC system (Dionex Ultimate 3000) equipped with an ESA Corona Charged Aerosol Detector (CAD). An extraction procedure adapted from Lalman and Bagley was performed prior to analysis. ¹Briefly, a 1 mL sample was removed from the fermentation culture and acidified using a 30% (v/v) H₂SO₄ solution. The fatty acids were extracted with 500 μL of a solvent system (n-hexane:MTBE=1:1 v/v), vortexed, and centrifuged in a microcentrifuge for 1 min. at 16,000×g. A 10 μL sample of the extract layer was injected on to the HPLC-CAD system and fatty acid analysis was performed using the following instrument parameters: column: Ascentis C8 15 cm×4.6 mm, 2.7 μm; guard column: Ascentis C8 0.5 cm×4.6 mm×2.7 μm; mobile phase A: 375:125:2 LC-MS grade methanol:deionized water:acetic acid; mobile phase B: 500:375:125:4 LC-MS grade acetonitrile:methanol:THF:acetic acid; flowrate: 0.8 mL/min; run program: 0-17.5 min. 75% A:25% B, 18.1-20.1 min. 60% A:40% B, 20.1-23.0 min. 75% A: 25% B; and column oven: 40° C. Fatty acid concentrations were calculated from calibration curves generated from analytical fatty acid calibration mixes analyzed under the same HPLC-CAD method.

Cell growth was measured by serial dilutions and plate counts.

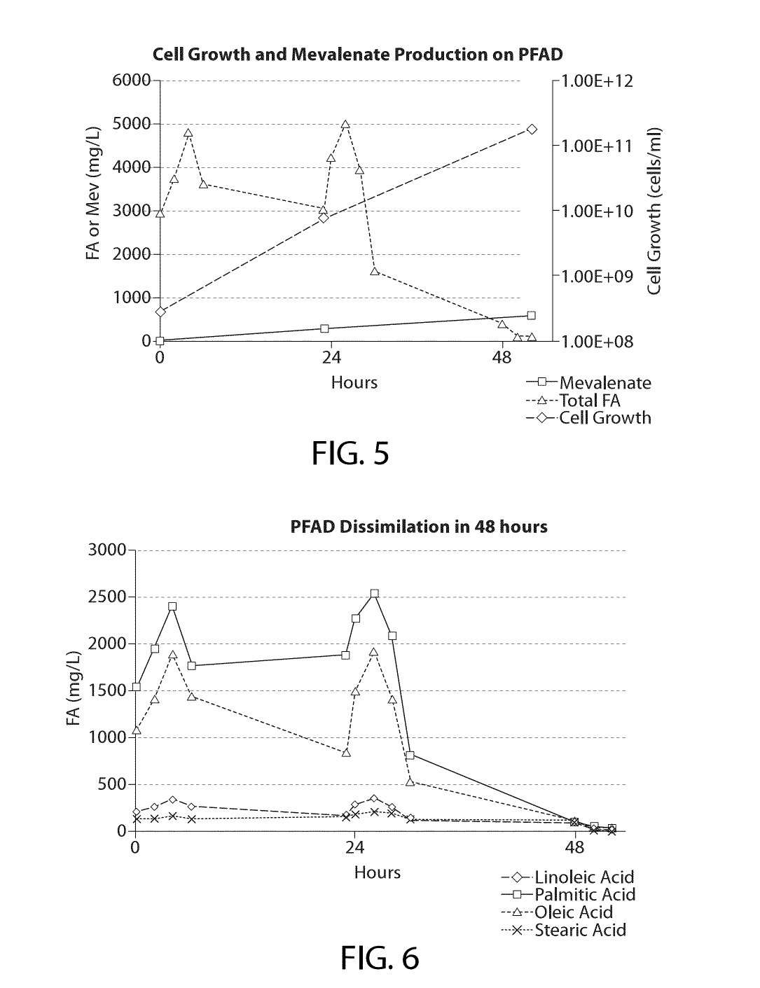

FIG. 5 shows that FA01 grows well on PFAD. FA01 doubled almost 10 generations in 48 hours. FIG. 5 also shows that FA01 produced 600 mg/l mevalonate in same period.

This example also illustrates that FA01 used all of the major constituents of PFAD, of which there are four: linoleic, oleic, stearic, and palmitic acid (FIG. 6). We observed that there was some preference for certain fatty acids under certain conditions (FIG. 6). First, unsaturated fatty acids were preferred in the first 24 hours while cell mass was low. But after sufficient cell mass was achieved, FA01 quickly consumed all four fatty acids simultaneously until they were depleted by 48 hours. Also, high concentrations of fatty acids were preferred over low concentrations of fatty acids. Once a species of fatty acid reached a certain low threshold, that species was ignored for higher concentrated species of fatty acids. This threshold created a baseline, for which the fatty acids were only consumed if they were above this threshold. This baseline of fatty acids was ignored until all other higher concentrated species of fatty acids were consumed, at which point, the cells dissimulated all species of fatty acids to less than the threshold. Despite these preferences, all fatty acid species were consumed by the end of the experiment.

We observed a mass transfer phenomenon in the solublization of fatty acids after each feeding. In this example, the cells were fed twice, at 0 and 23 hour time-points, during the course of the experiment. At both feedings, we observed an increase of soluble PFAD in the media over a period of four hours (FIG. 6). This phenomenon is reflective of the mass transfer rate of insoluble fatty acids to soluble fatty acids.

### Example 6

This example demonstrates that FA01 will continue to dissimilate PFAD after two weeks of growth with periods of starvations.

E. coli FA01, (strain MG 1655: ATCC700926), transformed with pGB 1008 (rep_{p15A}, Cm^{R}, tetR, P_{LtetO-1}, mvaES_{Ef,optEc}, T1), was culture in 400 ml of sterile minimal media in ½L bioreactors. Ingredients for the minimal media were as follows: 660 mg/l ammonium sulfate, 1.2 g/l sodium phosphate dibasic, 3 g/l ammonium chloride, 0.25 g/l potassium sulfate, 4 g/l magnesium chloride hexahydrate, 30 mg/l iron sulfate heptahydrate, 700 mg/l calcium chloride dihydrate, 0.173 mg/l sodium selenite, 0.004 mg/l ammonium molybdate tetrahydrate, 0.025 mg/L boric acid, 0.007 mg/l cobalt chloride hexahydrate, 0.003 mg/l copper (II) sulfate pentahydrate, 0.016 mg/l manganese chloride tetrahydrate, 0.003 mg/l zinc sulfate heptahydrate.

The minimal media was supplemented with hot PFAD (density 0.9036 g/ml) that was atomized through a 26-gauge needle. We cultured FA01 at 37° C. with a pH of 6.3 that was maintained with 5M NaOH. This experiment was under microaerobic conditions with an oxygen transfer rate coefficient of K_{L}A=60^{hr-1}. 20 ug/ml chloramphenicol was added to maintain selection of FA01 cells transformed with the pGB 1008 plasmid. Plasmid pGB 1008 was induced with 100 ug/L anhydrotetracycline.

Isolated colonies of FA01 were obtained by streaking cells onto LB agar plates from glycerol stocks. One isolated colony was collected by loop and used to seed 40 ml LB in a 250 ml baffle shake flask, which served as precultures for experiments. 20 ug/ml chloramphenicol was added to the shake flasks to maintain selection of FA01 cells transformed with the pGB 1008 plasmid. Precultures grew overnight at 37° C. with shaking at 175 rpm. Grown precultures were washed in sterile minimal medium salts before being added to the ½ L bioreactors with sufficient cells for a start OD₆₀₀ of 0.1.

For general metabolites, cultures were analyzed for liquid fermentation products by injecting 10 μL samples on to a HPLC (LC-10AD vp, Shimadzu, Kyoto, Japan) using a Rezex ROA-Organic Acid H⁺ (8%) column (Phenomenex, Torrance, Calif.) at 65° C., with a 2.5 mM H₂SO₄ mobile phase (isocratic, 0.6 mL/min), and ultraviolet (UV, SPD-10A vp, 280 nm) and refractive index detectors (RID-10A). All samples were filtered through a 0.22 μm polyvinylidene fluoride syringe filter (Millipore) prior to HPLC analysis.

Fatty acids were analyzed using a HPLC system (Dionex Ultimate 3000) equipped with an ESA Corona Charged Aerosol Detector (CAD). An extraction procedure adapted from Lalman and Bagley was performed prior to analysis [Lalman, Jerald A. and Bagley, David M., Journal of the American Oil Chemists' Society, Vol. 81, no. 2 (2004) 105-110]. Briefly, a 1 mL sample was removed from the fermentation culture and acidified using a 30% (v/v) H₂SO₄ solution. The fatty acids were extracted with 500 μL of a solvent system (n-hexane:MTBE=1:1 v/v), vortexed, and centrifuged in a microcentrifuge for 1 min. at 16,000×g. A 10 μL sample of the extract layer was injected on to the HPLC-CAD system and fatty acid analysis was performed using the following instrument parameters: column: Ascentis C8 15 cm×4.6 mm, 2.7 μm; guard column: Ascentis C8 0.5 cm×4.6 mm×2.7 μm; mobile phase A: 375:125:2 LC-MS grade methanol:deionized water:acetic acid; mobile phase B: 500:375:125:4 LC-MS grade acetonitrile:methanol:THF:acetic acid; flowrate: 0.8 mL/min; run program: 0-17.5 min. 75% A:25% B, 18.1-20.1 min. 60% A:40% B, 20.1-23.0 min. 75% A: 25% B; and column oven: 40° C. Fatty acid concentrations were calculated from calibration curves generated from analytical fatty acid calibration mixes analyzed under the same HPLC-CAD method.

Cell growth was measured by serial dilutions and plate counts.

In this example, cells were fed PFAD in varied concentrations every 24 hours for the first three days. Cells were then starved for four days. After which, feeding resumed and cells were fed again every 24 hours for the next three days.

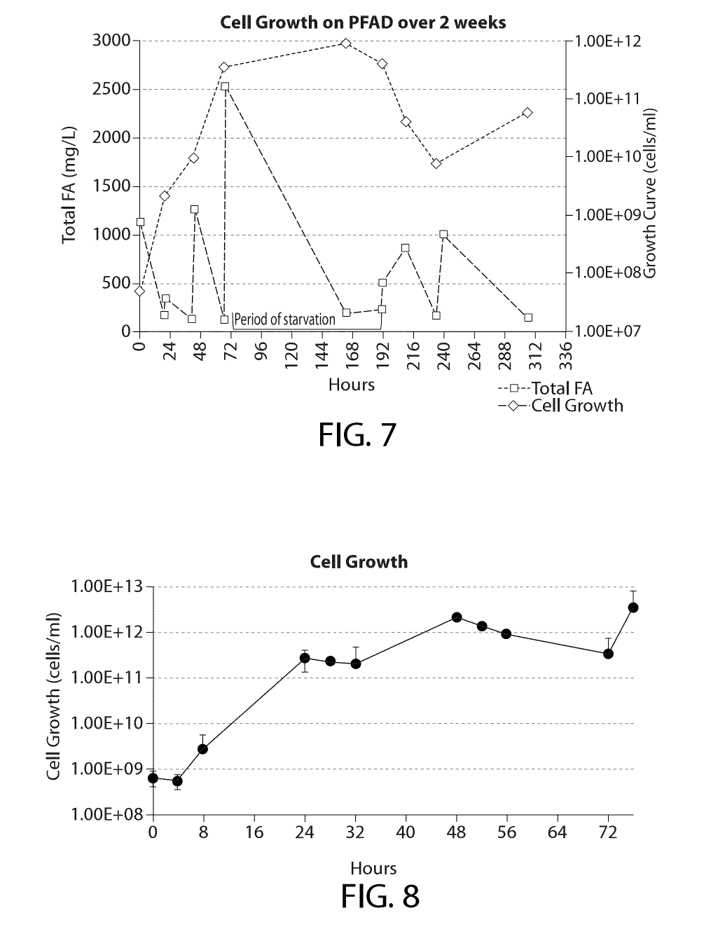

FIG. 7 shows that the cells still continued to dissimilate PFAD after 8-10 days of growth and after four days of starvation. The cells doubled almost 14 times over 7 days. Cells started to die over the next three days, but then started to recover by the end of the experiment.

### Example 7

In this example, we show that we achieved a growth rate of 1.34×10¹⁰ cells/ml/hr, and produced 2.13 g/l mevalonate in 72 hours at a rate of 0.03 g/l/h.

E. coli FA01, (strain MG 1655: ATCC700926), transformed with pGB 1008 (rep_{p15A}, Cm^{R}, tetR, P_{LtetO-1}, MvaES_{Ef,optEc}, T1), was cultured in 400 ml of sterile minimal media in ½L bioreactors. Ingredients for the minimal media were as follows: 660 mg/l ammonium sulfate, 1.2 g/l sodium phosphate dibasic, 3 g/l ammonium chloride, 0.25 g/l potassium sulfate, 4 g/l magnesium chloride hexahydrate, 30 mg/l iron sulfate heptahydrate, 700 mg/l calcium chloride dihydrate, 0.173 mg/l sodium selenite, 0.004 mg/l ammonium molybdate tetrahydrate, 0.025 mg/L boric acid, 0.007 mg/l cobalt chloride hexahydrate, 0.003 mg/l copper (II) sulfate pentahydrate, 0.016 mg/l manganese chloride tetrahydrate, 0.003 mg/l zinc sulfate heptahydrate. The minimal media was supplemented with hot PFAD (density 0.9036 g/ml) that was atomized through a 26-gauge needle. We added 1 ml of PFAD at 0, 8, 24 and 28 hours. We added 2 ml of PFAD at 32, 48, 52, 56, and 72 hours. We cultured FA01 at 37° C. with a pH of 6.3 that was maintained with 5M NaOH. This experiment was under very microaerobic conditions. Air was pumped only through the headspace of the bioreactors. 20 ug/ml chloramphenicol was added to maintain selection of FA01 cells transformed with the pGB1008.

Isolated colonies of FA01 were obtained by streaking cells onto LB agar plates from glycerol stocks. One isolated colony was collected by loop and used to seed 40 ml LB in a 250 ml baffle shake flask, which served as precultures for experiments. 20 ug/ml chloramphenicol was added to the shake flasks to maintain selection of FA01 cells transformed with the pGB1008 plasmid. Precultures grew overnight at 37° C. with shaking at 175 rpm. Grown precultures were washed in sterile minimal medium salts before being added to the ½L bioreactors with sufficient cells for a start OD₆₀₀ of 0.1.

See previous examples for how we measure mevalonate, cell growth, and residual fatty acids.

In this example, we show increased growth rates of FA01 when fed PFAD. In the first 24 hours, the cells grew at a rate of 1.34×10¹⁰ cell/ml/hr (FIG. 8). This equates to 8.73 doublings in the first 24 hours or 0.36 doublings per hour or one doubling every 2.75 hours. In the first 48 hours, the cells grew at a rate of 5.04×10¹⁰ cells/ml/hr (FIG. 8). This equates to 11.77 doubling in the first 48 hours or 0.25 doublings per hour or one doubling every 4.1 hours.

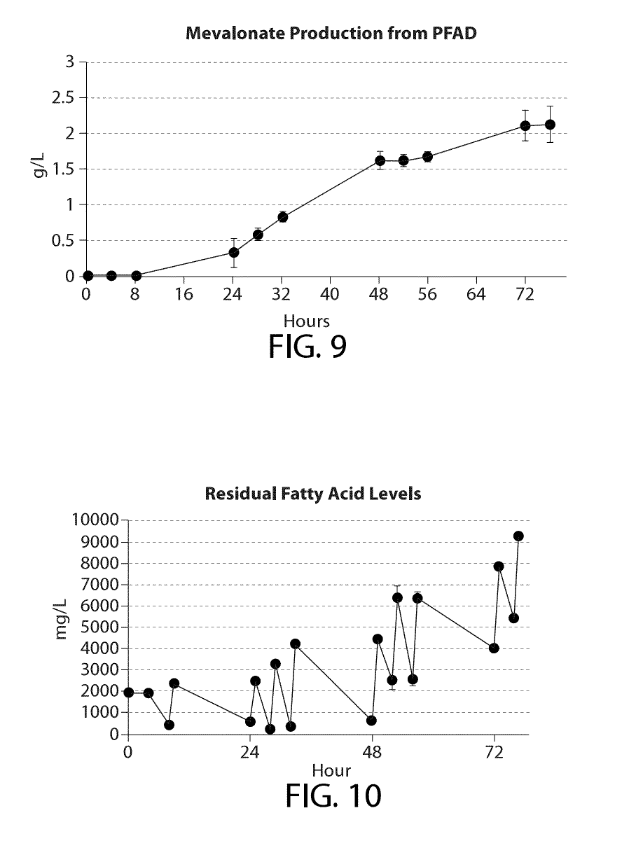

In this example, we also produced 2.13 g/l mevalonate in 72 hours at a rate of 0.03 g/l/h (FIG. 9), and we consumed an approximate total of 125 g/l PFAD in 72 hours (FIG. 10).

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A method of producing bioproducts from a feedstock, the method comprising:
a) providing a feedstock comprising fats, oils and fractions thereof;
b) heating the feedstock to a temperature that is above the melting point of the fats, oils and fractions thereof contained in the feedstock;
c) adding the feedstock of step (b) to fermentation media while mixing wherein the fermentation comprises at least one biocatalyst capable of fermentation in the presence of the feedstock, and wherein the feedstock forms a stable dispersion upon addition to the fermentation media with mixing;
d) fermenting the feedstock in the fermentation media while mixing to produce at least one bioproduct; and
e) optionally separating bioproduct, biocatalyst or feedstock from the fermentation media during fermentation or after fermentation has been completed.

2. The method of claim 1, wherein the fermentation media and the feedstock are free of exogenous surfactants.

3. The method of claim 1, wherein the fermentation media comprising the feedstock of step (c) are contained in a bioreactor for fermentation.

4. The method of claim 1, wherein the biocatalyst is a microorganism selected from mesophiles, thermophiles or both.

5. The method of claim 1, wherein the fermentation media of step (c) is at a temperature below the melting point of the fats, oils and fractions thereof contained in the feedstock when the feedstock is added.

6. The method of claim 1, wherein the fermentation media of step (c) is at a temperature above the melting point of fats, oils and fractions thereof contained in the feedstock when the feedstock is added.

7. The method of claim 5, further comprising the steps of raising the temperature of the fermentation media at the end of the fermentation process and separating feedstock that has not undergone bioconversion, biocatalyst, and bioproduct from the fermentation media.

8. The method of claim 1, wherein the fermentation media of step (c) is at a temperature between 25° C. to 70° C. when the feedstock is added.

9. The method of claim 1, wherein the fermentation media of step (c) is at a temperature of about 50° C. or higher when the heated feedstock is added.

10. The method of claim 1, wherein the fermentation media of step (c) is at a temperature of about 37° C. or higher when the heated feedstock is added.

11. The method of claim 1, wherein the fats, oils and fractions thereof contained in the feed stock are by-products of an industrial process.

12. The method of claim 11, wherein the industrial process comprises bio-fuel manufacture, fat saponification, alcoholic beverages manufacture, production of vegetable oils or processes used in the oleochemicals industry and processes used in the oil refining industry.

13. The method of claim 12, wherein the vegetable oil is palm oil.

14. The method of claim 1, further comprising adding a lipase to the feedstock prior to adding the feedstock to the fermentation media of step (c).

15. The method of claim 1, further comprising adding a lipase to the fermentation media simultaneously with the addition of the feedstock to the fermentation media of step (c).

16. The method of claim 1, wherein the biocatalyst of step (c) is a microorganism that over expresses a lipase.

17. The method of claim 1, further comprising the step of separating the biocatalyst from unfermented feedstock after the completion of the fermenting process of step (d).

18. The method of claim 1, wherein the feedstock comprises palm acid oil (PAO) and palm fatty acid distillate (PFAD).

19. The method of claim 18, wherein the PAO or PFAD comprise at least about 20% by weight of the feedstock.

20. The method of claim 1, wherein the fats, oils or fractions thereof comprise at least about 20% by weight of the feedstock.

21. The method of claim 1, wherein the heated feedstock in claim 1 step (b) is:
i) heated to a temperature greater than above about 45° C. or above about 48° C., or above about 50° C., or above about 52° C., or above about 55° C., or above about 58° C., or above about 60° C., or above about 70° C., or above about 80° C., or above about 100° C.;
ii) atomized at a temperature above about 45° C. or above about 48° C., or above about 50° C., or above about 52° C., or above about 55° C., or above about 58° C., or above about 60° C., or above about 70° C., or above about 80° C., or above about 100° C.; and,
wherein the temperature of the fermentation media of steps (c) or (d) in is between about 15° C. and 45° C., preferably between about 25° C. and 40° C.

22. The method of claim 21, wherein the heated feedstock comprises about 0.50 to about 1.5 g/ml PFAD.

23. The method of claim 22, wherein about 0.5 to about 100 part by volume of the heated feedstock is added to about 400 parts by volume of fermentation media in about six to eight hour time intervals of fermentation, and optionally an additional 0.5 to about 100 parts of the heated feedstock is added to the fermentation media at about 32, 48, 52, 56, and 72 hours of fermentation.

24. The method of according to claim 21, wherein fermentation occurs under microaerobic conditions.

25. A method of producing bioproducts from a feedstock, the method comprising:
a) providing a feedstock comprising fats, oils and fractions thereof wherein at least a portion of the fats, oils and fractions thereof are in semi solid, or solid form;
b) adding the feedstock of step (a) to fermentation media and mixing, wherein the fermentation media comprises at least one biocatalyst capable of fermentation in the presence of the feedstock, wherein the fermentation media is at a temperature above the melting point of the fats, oils and fractions thereof contained in the feedstock, and wherein the feedstock forms a stable dispersion upon addition to the fermentation media;
c) fermenting the feedstock in the fermentation media while mixing to produce at least one bioproduct; and
d) optionally separating bioproduct, biocatalyst or feedstock from the fermentation media during fermentation or after fermentation has been completed.

26-28. (canceled)

29. A method of producing bioproducts from a feedstock that is the by-product of an industrial process, the method comprising:
a) providing a feedstock comprising by-products derived from an industrial process wherein the by-products comprises at least about 20% fats, oils and fractions thereof;
b) heating the feedstock to a temperature that is above the melting point of the fats and oils contained in the feedstock;
c) adding the heated feedstock of step (b) to fermentation media and mixing wherein the fermentation media comprises at least one biocatalyst capable of fermentation in the presence of the feedstock, and wherein the feedstock forms a stable dispersion upon addition to the fermentation media;
d) fermenting the feedstock in the fermentation media while mixing to produce at least one bioproduct; and
e) optionally separating bioproduct, biocatalyst or feedstock from the fermentation media during fermentation or after fermentation has been completed.

30-36. (canceled)

37. A method of producing bioproducts from a feedstock comprising the by-products of an industrial process, the method comprising:
a) providing a feedstock comprising by-products derived from an industrial process wherein the by-products comprise fats, oils and fractions thereof and wherein at least a portion of the fats, oils and fractions thereof are semi-solids or solids;
b) adding the feedstock of step (a) to fermentation media while mixing wherein the fermentation media comprises at least one biocatalyst capable of fermentation in the presence of the feedstock, wherein the fermentation media is at a temperature above the melting point of the fats, oils and fractions thereof contained in the feedstock, and wherein the feedstock forms a stable dispersion upon addition to the fermentation media;
c) fermenting the feedstock in the fermentation media while mixing at a temperature that is higher than the melting points of the fats oils and fractions thereof contained in the feedstock to produce at least one bioproduct; and
d) optionally separating bioproduct, biocatalyst or feedstock from the fermentation media during fermentation or after fermentation has been completed.

38-39. (canceled)

40. A composition for use in biofermentation comprising a feedstock, wherein the feedstock comprises at least about 20% by weight of fats, oils or fractions thereof, and wherein the feedstock is stably dispersed in fermentation media when mixed in the fermentation media, wherein the fermentation media comprises at least one biocatalyst capable of using the feedstock as a substrate for producing at least one bioproduct and wherein the composition is free of surfactants.

41-46. (canceled)

47. A composition for use in biofermentation comprising a feedstock, wherein the feedstock comprises PFAD, and wherein the PFAD is stably dispersed in fermentation media when mixed in the fermentation media, wherein the fermentation media comprises at least one biocatalyst capable of using the feedstock as a substrate for producing at least one bioproduct and wherein the composition is free of surfactants.

48-54. (canceled)

55. A suspension or dispersion comprising PFAD or PAO wherein said suspension or dispersion is produced by heating a composition comprising PFAD or PAO above the melting point of said PFAD or PAO and atomizing said heated composition comprising PFAD or PAO and adding said atomized composition comprising PFAD or PAO to an aqueous media.

56-61. (canceled)

62. A method for producing a substantially homogeneous suspension or dispersion comprising a semi-solid or fat substance wherein a composition comprising said semi-solid substance or fat composition is heated above the melting point of said semi-solid or fat substance and added to an aqueous media with optional mixing.

63-66. (canceled)
